(19) United States
(12) Patent Application Publication
(10) Pub. No.: US 2005/0233376 A1
(43) Pub. Date: Oct. 20, 2005

(54) Biochip scanner device

(75) Inventors: Jiann-Hua Wang, Taipei (TW); Hui-Ju Chen, Dongshan Township (TW); Tzu-Chiang Wu, Miaoli City (TW); Chien-Ho Chuang, Kaohsiung (TW); Tsung-Kai Chuang, Tainan City (TW)

Correspondence Address: BIRCH STEWART KOLASCH & BIRCH, PO BOX 747, FALLS CHURCH, VA, 22040-0747, US

(73) Assignee: Kaiwood Technology Co., Ltd.

(21) Appl. No.: 11/166,238

(22) Filed: Jun. 27, 2005

Related U.S. Application Data
(63) Continuation-in-part of application No. 10/760,465, filed on Jan. 21, 2004.

(51) Int. Cl. C12Q 1/68; C12M 1/34

(52) U.S. Cl. 435/6; 435/287.2; 435/288.7; 435/808

(57) ABSTRACT

A biochip scanner device includes a light source for emitting a light beam; a light processing unit for focusing the light beam onto the biochip to excite fluorescence from a sample spot on the biochip; a filter for filtering off the light beam from the light source and the scattered light from the surface on the biochip; a photomultiplier tube (PMT) for detecting and converting the fluorescence into a current signal; and an output device for outputting/displaying the current signal detected by the PMT. The output device controls the platform to match the pattern of the biochip. No conversion of the output signal of the output device into image data is needed. A real-time analysis proceeds while samples are being scanned on the biochip. The biochip scanner device of the present invention reads the current signal from PMT directly without processing it into image data and setting lens before the PMT is no longer needed. As a result, the structure of the device is simplified and the cost for production is reduced.

### CROSS REFERENCE TO RELATED APPLICATIONS

This invention is a continuation in part of U.S. Ser. No. 10/760,465, filed on Jan. 21, 2004, and entitled “BIOCHIP SCANNER DEVICE”.

### BACKGROUND OF THE INVENTION

1. Field of the Invention

The present invention relates to a biochip scanner device, particularly to a biochip scanner device to detect fluorescent signal emitting from biochips.

2. The Prior Arts

A “working draft” of the human sequence produced by the Human Genome Project published in Nature (15 Feb., 2001), simultaneously with a companion publication of the human sequence generated by Celera Genomics Corporation (Science, 16 Feb., 2001). The next important goal is determining the function of the genes. To accelerate the progress of the related research, high-throughput tools for efficient analysis are available. A biochip which contains results of mass samples expressed on the surface of the small solid carrier, is a useful analytic tool. Biochips can be employed in gene expression, drug selection and disease diagnosis in both basic research and clinical application fields.

The DNA chip is the majority type of biochip. FIG. 1 illustrates a detection method of the biochip. A number of known DNA fragments (2) are placed on a surface of a solid carrier to form a DNA chip (1). Generally, DNA probes (2) are arranged in an array called DNA microarray. Unknown DNA fragments (3), target DNA, are labeled with fluorescent dyes. The DNA chip (1) is then hybridized with the target DNA (3). After washing, only DNA fragments, which hybridized with the DNA probes are left on the DNA chip (1). A biochip reader can then read and detect the fluorescence excited from the fluorescent dyes.

FIG. 2 shows a conventional biochip reader. In the biochip reader (4), beams of light emitted from a laser light source (40) which pass through a focusing lens (41) and are reflect by a beam splitter (42), and then further pass through the focusing lens (43) to a surface of the biochip (44) that is deposited in the reader (4). The fluorescent dyes on the biochip (44) are excited by the beams and in turn emit the fluorescence (45). The fluorescence (45) so emitted passes through the focusing lens (43), the beam splitter (42), and the focusing lens (46) in sequence. The fluorescence (45) passes through a filter (47). The fluorescence signal is thus applied to a photomultiplier tube (PMT) (48), which converts the optic signal into an amplified electrical signal. The electrical signal is fed to a computer (49) and processed to form an image data.

Typically, a biochip analyzer is a high-resolution imagine instrument, which outputs a digital image of biochip to exactly show the relative fluorescence intensities of the sites immobilized samples. Such image processing steps including locating spot centers, classification of pixels either as signal or background, and calculating signal intensity, background and quality measuring are complicate and time-consuming. With increasing complexity of biochips, the software for processing the fluorescence data becomes increasing intricate and rather demanding in terms of computer memory and processor speed, and the cost for manufacturing a biochip reader becomes higher. To overcome the disadvantages of the conventional biochip reader, U.S. Pat. No. 6,407,395 provides a portable biochip scanner device, which digitalizes the signals from PMT with an analog-to-digital converter (ADC) and processes the digital signals to display or output the integral signals representing the fluorescence intensity on the biochip array. However, the data processing including digital filtering and signal integration is also time-consuming, and simultaneous scanning and analysis is not achievable since the data processing is started after the scanning step.

There are still other disadvantages in the conventional biochip reader. For example, signal deviation between biochip and output image is not avoidable when the continuous detected PMT signal is converted into a digital image of biochip. Furthermore, the conventional biochip reader uses a high-speed motor to continuously scan a biochip and read the signal with a scanning pathway including the area with or without sample spots. The scanning and reading operations take a lot of time, the processing and analysis for such a lot of data are time-consuming, and the computer memory and processor speed are more demanded. And the signals from the area without sample spots are a noise source which can interference the fluorescent signal desired.

U.S. Pat. No. 6,407,395 disclosed a discrete scanning (or raw scanning) technique exclusively the rows of a biochip array, and the scanner allows the beam focal spot being adjusted to match the size of the array element (sample spot). The scanner moves a biochip for directing the beam focal spot to illuminates the sample spot one at a time and collects the fluorescence. However, it is difficult to adjust the beam focal spot to match the size of sample spot if the sample spots are not uniform on the biochip, and the spot-by-spot illuminating process is also time-consuming.

Moreover, in general, manually or automatically circling each sample spot on computer to analyze is carried out after scanning all the sample spots, but the circling operation wastes a lot of time. Also, the conventional biochip reader does not provide the final result until all the steps of sample scanning and image processing are finished, and the assay result cannot be shown simultaneously.

Besides, some focusing lenses are usually used for focusing the laser beam of excitation radiation onto the biochip or focusing the illuminated fluorescence light of the assayed target toward detector in a biochip reader. For example, U.S. Pat. Nos. 6,407,395, 6,563,584, and 6,310,687 use a focusing lens for focusing the illuminated fluorescence light of the assayed target toward detector. U.S. Pat. No. 6,407,395 and U.S. Pub. No. 2003/0020022 use an objective lens (generally, an objective lens includes a set of a plurality of lenses) for focusing the laser beam of excitation radiation onto the biochip. Although the focusing lenses is effective to collect the fluorescence light illuminated from the assayed target, the cost for manufacturing a biochip reader is also increased with the number of used lenses.

### SUMMARY OF THE INVENTION

A primary object of the present invention is to provide a biochip scanner device that overcomes the above-mentioned disadvantages and allows for doing a real-time analysis when simultaneously scanning. Scanning all samples on a biochip and forming image data for analysis are no longer necessary. Therefore, easy and efficient operation is realized.

The second object of the invention is to provide a biochip scanner device that reads electrical signal (such as current signal) from a photomultiplier tube (PMT) directly without converting the electrical signal into a digital image data first and thus eliminating errors that occur in the conversion processing.

Furthermore, there is no longer a need for setting lens before the PMT. As a result, the structure of the device is simplified and the production cost is reduced.

In order to realize the foregoing objects, a biochip scanner device of the present invention comprises: a light source for emitting a light beam; a light processing unit for focusing the light beam onto the biochip to excite fluorescence from a sample spot on the biochip; a filter for filtering off the light beam from the light source; a photomultiplier tube (PMT) for detecting and converting the fluorescence into a current signal; and an output device, which comprises a first set of parameters for directly reading the current signal detected by the PMT without converting the current signal into a digital image data.

The aforementioned light processing unit may comprises: a beam splitter for redirecting the light beam to pass through a focusing lens, which focuses the light beam onto the biochip and excites fluorescence from a sample spot on the biochip. The focusing lens herein is just a single focusing lens but an objective lens, and its cost is lower than the objective lens.

Preferably, a PMT detector with large detected area is used to collect the fluorescence light completely even the light is not collimated sufficiently and scattered. Moreover, the distance between the PMT detector and the focusing lens is preferably as close as possible to decrease the scattered angle of the fluorescence light. Thus, only a single focusing lens is need to focus the light beam onto the biochip, and the cost of the biochip reader can be reduced.

Besides, additional focusing lens may be set between the light source and the beam splitter to enhance the focusing effect.

The aforementioned parameters may be a set of parameters reading, transferring, and outputting or displaying the current signal from PMT on the output device directly. Without the steps for image processing, the biochip reader of the present invention achieves simultaneous scanning and analysis. Furthermore, the parameters are so simple that the cost of biochip reader is reduced.

Preferably, the present biochip scanner device comprising a second set of parameters controlling the directions of movement of the platform, moving the platform according to a predetermined pathway corresponding to the sample spot one by one, and starting signal reading just on the sample spot but stopping signal reading in an area without the sample spot. The predetermined pathway can be determined according to the array of sample spot on the biochip. The predetermined pathway including the moving distance and distance corresponding to the positions of sample spot can be input into the computer previously, the computer controls the directions of movement of the platform, moves the sample spot to let each at the precisely position for PMT detection one by one. Contrast to the conventional biochip reader, only the signal from the sample spot but not the area without the sample spot is read and taken in the present biochip scanner device. Accordingly, it can reduce the noise interference from the background on biochip and saving a lot of time for scanning process. And the step for spot circling is not necessary to shorten the analyzed time.

Furthermore, a psudo-image data may be optionally derivated from the signal detected by the PMT. The psudo-image data but not a digital image is consisted of a color spot array, the color density of the color spots reflects the current intensity from the PMT. The psudo-image data can provide a rough reference for user to browse the analysis result. The signal intensity of each sample still comes from the signal detected by the PMT and therefore no errors arise as in the process of converting the electrical signal into image data in the conventional biochip.

A real-time analysis proceeds while samples are being scanned on the biochip. Fluorescence of each sample is collected by the PMT, which converts the fluorescence signal to an electrical signal. Setting lens before the PMT is no longer needed for unnecessary converting the electrical signal into image data.

For more detailed information regarding advantages and features of the present invention, examples of preferred embodiments will be described below with reference to the annexed drawings. It is to be understood that both the foregoing general description and the following detailed description are exemplary and explanatory and are intended to provide further explanation of the invention as claimed.

### BRIEF DESCRIPTION OF THE DRAWINGS

The related drawings in connection with the detailed description of the present invention to be made later are described briefly as follows, in which:

FIG. 1 illustrates DNA chip detection system in the prior art;

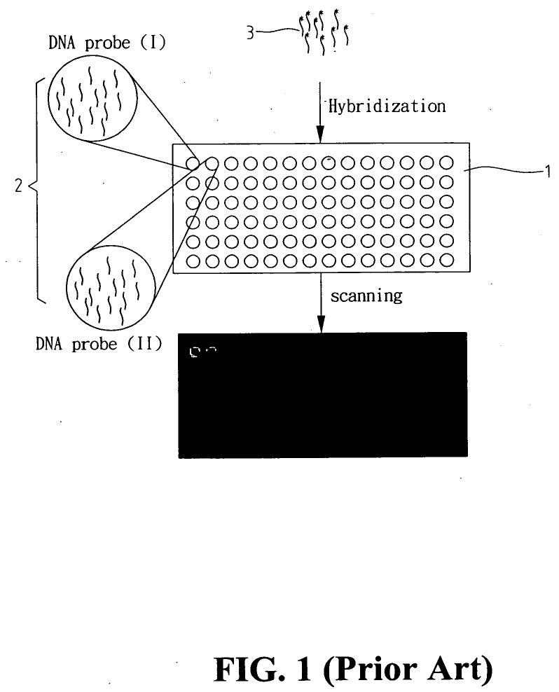

FIG. 2 is a schematic view showing one example of a conventional biochip reader;

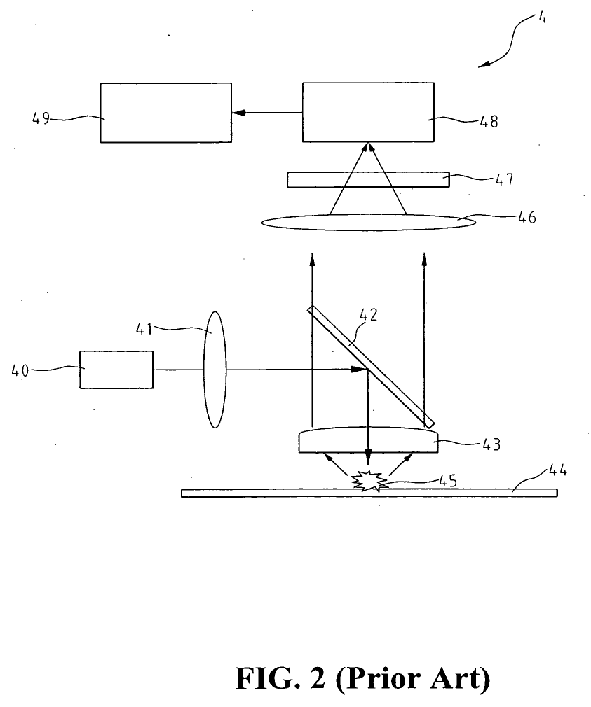

FIG. 3 is a schematic view showing one embodiment of the biochip scanner device of the present invention;

FIG. 4 is a diagram illustrating a moving way of the platform controlled by the computer in accordance with another embodiment of the biochip scanner device of the present invention;

FIG. 5 shows real-time output signal obtained by the biochip scanner device of the present invention; and

FIG. 6 shows comparison image data of samples on the biochip obtained by the biochip scanner device of the present invention.

### DETAILED DESCRIPTION OF THE PREFERRED EMBODIMENT

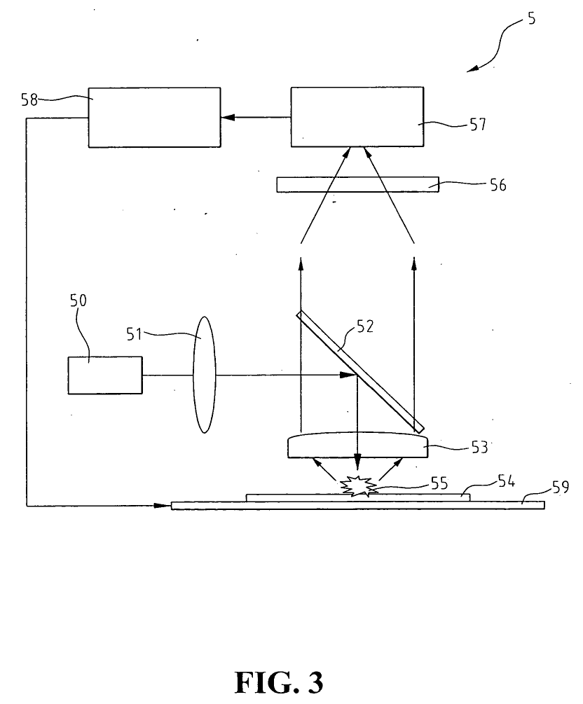

A preferred embodiment of the present invention will now be described in detail below with reference to the accompanying drawings. FIG. 3 shows an embodiment of a biochip scanner device in accordance with the present invention. The biochip scanner device, generally designated with reference (5), comprises a light source (50) for emitting a light beam. A beam splitter (52) redirects the light beam through a focusing lens (53), which focuses the light beam onto a biochip (54) to cause excitement of fluorescence (55) from a sample spot deposited on the biochip (54). A filter (56) filters off the light beam from the light source and the scattered light from the surface on the biochip (50). A photomultiplier tube (PMT) (57) detects the fluorescence and converts the fluorescence into an electrical signal. An output device (58) receives and shows the electrical signal detected by the PMT. Additional focusing lens (51) may be set between the light source (50) and the beam splitter (52) to enhance the focus effect.

A biochip is placed on a platform (59) when analyzed by the biochip scanner device of the present invention. The platform (59) is movable in two different directions, for example X and Y directions, under the control of a computer (58). In scanning, a light beam from a laser source (50) passes through the focusing lens (51) and reaches a surface of the biochip (54). Fluorescence (55) is excited from the sample spot on the biochip (54). The fluorescence (55) passes through the beam splitter (52). The light beam from the light source and the scattered light from the surface on the biochip are filtered out by the filter (56), the fluorescence is clearly detected by the PMT (57) and converted into a current signal. The current signal is transmitted to an output device (58). The signal is output/display directly from the output device (58). The output device (58) may be a computer which comprises a first set of parameters for directly reading the current signal detected by the photomultiplier tube, and the parameters read, transfer, and output or display the current signal from PMT (57) on the output device (58) directly. Converting the current signal into a digital imagine data is not necessary because the current signal from PMT (57) can reflect the fluorescence intensity from each sample spot. The current signal from PMT (57) can be simultaneously output on the output device with sample spot scanning.

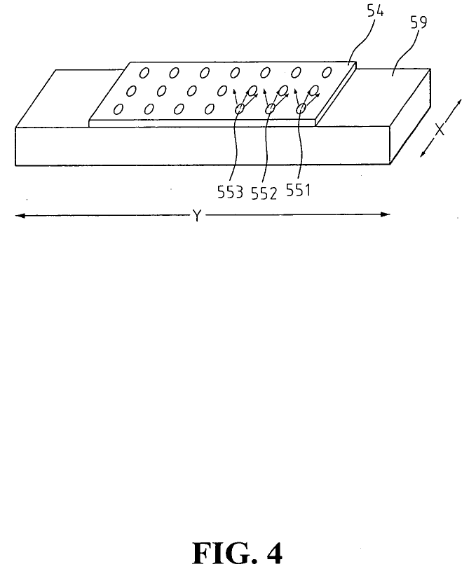

As shown in FIG. 4, the output device (58) may comprises a second set of parameters for controlling the directions of movement of the platform (59), moving the platform (59) according to a predetermined pathway corresponding to the sample spot (such as 551, 552 and 553) one by one, and starting signal reading just on the sample spot but stopping signal reading in an area without the sample spot. The predetermined pathway can be determined according to the array of sample spot on the biochip and matches the pattern of the biochip. The predetermined pathway including the moving distance and distance corresponding to the positions of sample spot can be input into the computer previously, the computer controls the directions of movement of the platform (59), moves the sample spot (such as 551, 552 and 553) to let each at the precisely position for PMT detection one by one.

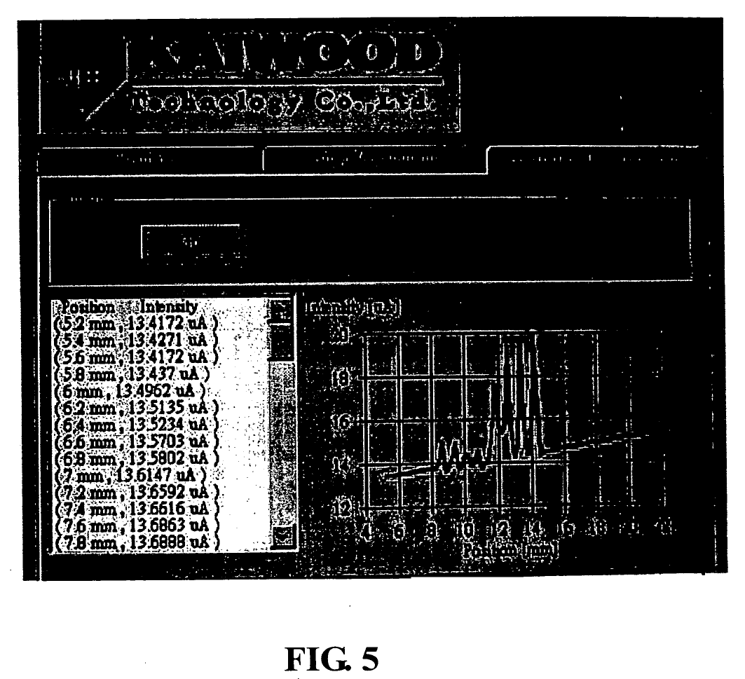

The output signal from the PMT is shown in FIG. 5. A real-time analysis proceeds while samples are being scanned on the biochip. The biochip scanner device of the present invention reads and displays the current signal from PMT directly without processing it into a digital image data.

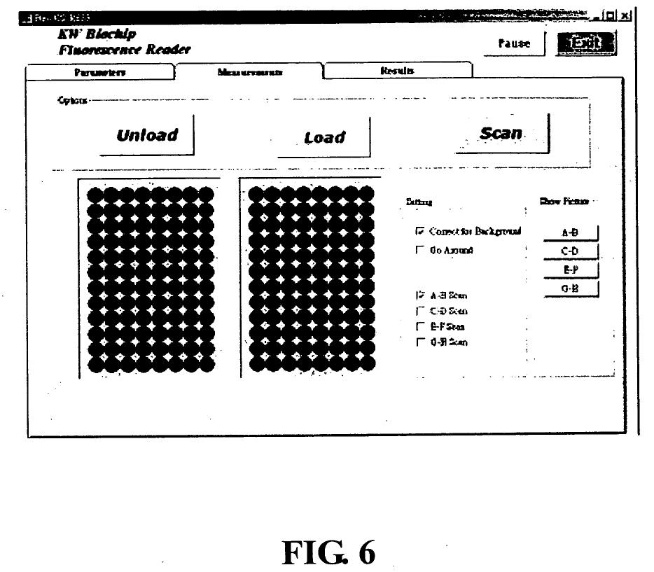

In addition, a psudo-image data as shown in FIG. 6 may be optionally derivated from the current signal detected by the PMT. The psudo-image data provides a rough reference for user to browse the analysis result. The signal intensity of each sample still comes from the signal detected by the PMT and therefore no errors arise as in the process of converting an electrical signal into image data in the conventional biochip.

1. A biochip scanner device for simultaneous scanning and analysis, comprising:
a light source, which emits a light beam;
a light processing unit, which focuses the light beam onto a biochip to excite fluorescence from a sample spot on the biochip;
a filter, which filters off the light beam from a light source and a scattered light from the surface on the biochip;
a photomultiplier tube, which detects and converts the fluorescence into a current signal; and
an output device, which comprises a first set of parameters for directly reading the current signal detected by the photomultiplier tube without converting the current signal into a digital imagine data;
wherein, the parameters read, transfer, and output or display the current signal from PMT on the output device directly.

2. The biochip scanner device according to claim 1, wherein the light processing unit comprises:
a beam splitter for redirecting the light beam through a focusing lens, which focuses the light beam onto the biochip and excites fluorescence from a sample spot on the biochip.

3. The biochip scanner device according to claim 2, wherein the light processing unit further comprises another focusing lens between the light source and the beam splitter to enhance the focus effect.

4. The biochip scanner device according to claim 1 further comprising a platform for holding the biochip and moving in two different directions.

5. The biochip scanner device according to claim 4, wherein the output device is a computer comprising a second set of parameters for controlling the directions of movement of the platform, moving the platform according to a predetermined pathway which is determined according to positions of the sample spots on the biochip, and starting signal reading just on the sample spot but stopping signal reading in an area without the sample spot.

6. The biochip scanner device according to claim 1, wherein the output device comprises at least one set of parameters for producing a psudo-image data comprising a color spot array according to the current signal detected by the photomultiplier tube.

7. A biochip scanner device for simultaneous scanning and analysis, comprising:
a light source, which emits a light beam;
a beam splitter for redirecting the light beam through a focusing lens, which focuses the light beam onto the biochip and excites fluorescence from a sample spot on the biochip;
a filter, which filters off the light beam from a light source and a scattered light from the surface on the biochip;
a photomultiplier tube, which detects and converts the fluorescence into a current signal; and
an output device, which comprises at least one set of parameters for directly reading the current signal detected by the photomultiplier tube without converting the current signal into a digital imagine data;
wherein, the parameters read, transfer, and output or display the current signal from PMT on the output device directly.

8. The biochip scanner device according to claim 7, further comprising another focusing lens between the light source and the beam splitter to enhance the focus effect.

9. The biochip scanner device according to claim 7, further comprising a platform for holding the biochip and moving in two different directions.

10. The biochip scanner device according to claim 9, wherein the output device is a computer comprising a second set of parameters for controlling the directions of movement of the platform, moving the platform according to a predetermined pathway which is determined according to positions of the sample spots on the biochip, and starting signal reading just on the sample spot but stopping signal reading in an area without the sample spot.

11. The biochip scanner device according to claim 7, wherein the output device comprises at least one set of parameters for producing a psudo-image data comprising a color spot array according to the signal detected by the photomultiplier tube.

12. A method of simultaneously scanning and analyzing samples on a biochip, comprising the steps of:
(a) placing a biochip having sample spot on a platform of a biochip scanner device according to claim 1;
(b) inputting a predetermined pathway which is determined according to positions of the sample spots on the biochip for controlling movement of the platform;
(c) scanning the biochip with a light beam from a laser source, wherein the light beam passes through focusing lens;
(d) exciting the fluorescence with the light beam;
(e) detecting the fluorescence with a photomultiplier tube;
(f) converting the fluorescence into a current signal;
(g) transmitting the current signal to an output device; and
(h) outputting the current signal on the output device.
